(19) United States
(12) Patent Application Publication
(10) Pub. No.: US 2015/0083146 A1
(43) Pub. Date: Mar. 26, 2015

(54) PERSONAL VAPORIZER LIQUID FOR EMULSIFYING OIL-SOLUBLE COMPOUNDS AND RESINS

(71) Applicant: Verso PV, LLC, Fort Collins, CO (US)

(72) Inventors: Maxwell L. Goldman, Fort Collins, CO (US); Lucas K. Marthaler, Fort Collins, CO (US); William Bjorncrantz, Stevensville, MI (US)

(21) Appl. No.: 14/494,234

(22) Filed: Sep. 23, 2014

Related U.S. Application Data
(60) Provisional application No. 61/881,336, filed on Sep. 23, 2013.

(51) Int. Cl. A24B 15/10; A24F 47/00; A61K 36/185; A61K 31/05; A61K 31/352

(52) U.S. Cl. CPC A24B 15/10; A61K 31/05; A61K 31/352; A61K 36/185; A24F 47/008
USPC 131/329; 131/352

(57) ABSTRACT

The present technology relates to liquid compositions for use in a personal vaporizer. More particularly, the present technology relates to liquid compositions for forming stable emulsions of oil-soluble compounds and resins, and to methods of administering vaporized oil-soluble compounds and resins to a subject by inhalation.

### CROSS-REFERENCE TO RELATED APPLICATIONS

This application claims priority to and the benefit of U.S. Provisional Application No. 61/881,336, filed Sep. 23, 2013.

### FIELD OF THE INVENTION

The present technology relates to liquid compositions for use in a personal vaporizer. More particularly, the present technology relates to liquid compositions for forming stable emulsions of oil-soluble compounds and resins, and to methods of administering vaporized oil-soluble compounds and resins to a subject by inhalation.

### BACKGROUND OF THE INVENTION

Personal vaporizers are devices that turn a liquid into a vapor so that a person can inhale the vapor. Many personal vaporizers are relatively small (pocket-sized or pen-sized), battery-powered, and use an atomizer or heater to vaporize the liquid. The personal vaporizer converts a liquid into a vapor that is inhaled. Personal vaporizers are inherently portable and come in a variety of forms.

An exemplary personal vaporizer is found in the Pen Kits available from Verso PV of Fort Collins, Colo. Pictures and instructions for use of the Verso personal vaporizer are shown in Appendix A. Additional examples of commercially available personal vaporizers include the liquid personal vaporizers from Atmos; the vaporizer pen from VapeToys; and many others. Personal vaporizers include electronic flameless vapor inhaler units that may simulate a cigarette.

Personal vaporizers are often used as alternatives to smoked tobacco products, such as cigarettes, cigars, or pipes. Inhaled doses of vapor provide a physical sensation similar to smoking. However, because a personal vaporizer is typically electrically powered, no tobacco, smoke, or combustion is usually involved in its operation. For portability, and to simulate the physical characteristics of a cigarette, cigar, or pipe, a personal vaporizer may be battery powered. In addition, a personal vaporizer may be loaded with a nicotine bearing substance and/or a medication bearing substance. The personal vaporizer may provide an inhaled dose of nicotine and/or medication by way of the heated and atomized substance. Thus, personal vaporizers are sometimes referred to as electronic cigarettes, or e-cigarettes. However personal vaporizers are not limited to delivering nicotine or acting as a cigarette substitute. To the contrary, they may be used to administer flavorants, medicinal agents, or other substances that are vaporized and then inhaled.

Personal vaporizers can be filled with liquid compositions to be vaporized. Such liquid compositions generally include a liquid base and one or more other components. Typical personal vaporizer liquid bases include propylene glycol, glycerol, or PEG-400, and they are primarily intended to function with the addition of water-soluble additives.

Besides delivering nicotine or being used as a cigarette substitute, personal vaporizers have been used to provide a vapor from solutions of actives (such as ingredients, flavors and/or medicinal compounds) in a base of propylene glycol, which is the standard liquid base for e-cigarettes. Propylene glycol is non-toxic and safe for daily consumption. When heated, propylene glycol produces a silky smooth vapor with a mildly sweet taste. When a solution of propylene glycol, actives, and flavorings are mixed and heated, the vapor becomes infused with the added ingredients, taking on a variety of flavors and therapeutic uses. Ingredients such as nicotine and flavorings such as “Peach Flavor” from LorAnn Oils have been used in liquid compositions for personal vaporizers.

However manufacturers of personal vaporizer liquids have had difficulty creating a stable emulsion containing oil-soluble compounds. The personal vaporizer liquids presently on the market typically have a bad taste and/or do not perform well, due to ill-suited food additives and unstable emulsions, respectively. Many products on the market use ethanol for emulsification, but this is not sufficiently effective and causes a bad taste at higher concentrations. Other emulsifiers will thermally decompose and produce carcinogenic byproducts and bad flavor.

Personal vaporizers can also be used for the delivery of cannabinoids or cannabis extracts. The National Institutes of Health (NIH) released a review of the scientific data concerning potential therapeutic uses for marijuana in 1997. In that review, the NIH found that marijuana may have beneficial medicinal effects and recommended that researchers develop alternative dosage forms for the drug, such as a “smoke free” inhaled delivery system. Various studies have documented therapeutically beneficial medicinal uses of the major active component of marijuana, delta-9-tetrahydrocannabinol (THC). See U.S. Pat. No. 6,713,048, entitled “Δ⁹ tetrahydrocannabinol (Δ⁹ THC) solution metered dose inhalers and methods of use”.

### SUMMARY OF THE INVENTION

The present compositions and methods provide a new and improved, ethanol-free, personal vaporizer liquid base for the emulsion of oil-soluble compounds, for use in a personal vaporizer. The present compositions and methods are superior in performance and taste with respect to personal vaporizer liquids for the emulsion of oil-soluble compounds. The present compositions and methods solve the problem of emulsifying oil-soluble compounds and/or resins for use in a personal vaporizer, without causing thermal decomposition products or without affecting the taste and potency of the vaporized material.

As one aspect of the present invention, a liquid composition as a stock solution for a personal vaporizer is provided. The liquid composition comprises a liquid base suitable for vaporization and inhalation; and a lipid solubilizer selected from the group consisting of a monoglyceride or diglyceride of one or more fatty acids or mixture thereof; an ester of monoglyceride or diglyceride of one or more fatty acids or mixture thereof; a propane-1,2-diol ester of one or more fatty acids or mixture thereof; a tri(C₁₋₈) alkyl ester of a C₂₋₈ organic acid or mixture thereof; and combinations thereof.

As another aspect of the present invention, a liquid composition for use in a personal vaporizer is provided. The liquid composition comprises a liquid base suitable for vaporization and inhalation; a lipid solubilizer selected from the group consisting of a monoglyceride or diglyceride of one or more fatty acids or mixture thereof; an ester of a monoglyceride or diglyceride of one or more fatty acids or mixture thereof; a propane-1,2-diol ester of one or more fatty acids or mixture thereof; a tri(C₁₋₈) alkyl ester of a C₂₋₈ organic acid or mixture thereof; and combinations thereof; and one or more oil-soluble compounds or resins or combinations thereof. The liquid composition is a stable emulsion.

As yet another aspect of the present invention, a method is provided for administering vaporized oil-soluble compounds and resins to a subject by inhalation. The method comprises vaporizing any of the liquid compositions described herein in a personal vaporizer. The method can also include dispensing the vaporized oil-soluble compounds or resins through a mouthpiece of the personal vaporizer. The method can also include inhalation of the vaporized oil-soluble compounds or resins. Where the vaporized oil-soluble compounds or resins are a medicinal agent, the method can also include treating a disease, disorder or condition with the vaporized oil-soluble compounds or resins.

As further aspects of the present invention, a container holding a liquid composition for a personal vaporizer is provided. The container holds a liquid composition that includes a liquid base suitable for vaporization and inhalation; a lipid solubilizer selected from the group consisting of a monoglyceride or diglyceride of one or more fatty acids or mixture thereof; an ester of a monoglyceride or diglyceride of one or more fatty acids or mixture thereof; a propane-1,2-diol ester of one or more fatty acids or mixture thereof; a tri(C₁₋₈) alkyl ester of a C₂₋₈ organic acid or mixture thereof; and combinations thereof; and one or more oil-soluble compounds or resins; wherein the liquid composition is a stable emulsion, wherein the container is adapted for attachment to a personal vaporizer. Also, the present invention provides a kit comprising a personal vaporizer, a battery charger, and the foregoing container with a liquid composition for a personal vaporizer.

Various embodiments are disclosed of the foregoing aspects of the present invention. In any of those aspects, the liquid base can be combined with a monoglyceride or diglyceride of one or more fatty acids (e.g., a E471 food additive such as monolaurin) or mixture thereof; an ester of a monoglyceride or diglyceride of one or more fatty acids or mixture thereof; a propane-1,2-diol ester of one or more fatty acids or mixture thereof. Alternatively or additionally, the liquid base can comprises a tri(C₁₋₈) alkyl ester of a C₂₋₄ organic acid (e.g., triethyl citrate). Preferably, the major portion of the liquid base is propylene glycol, and the composition is essentially free of ethanol.

In a liquid composition suitable for use in a personal vaporizer, the one or more oil-soluble compounds comprises a flavorant or a medicinal agent. For example, medicinal agent can comprise a cannabinoid, a cannabis extract or a combination thereof, such as cannabidiol (CBD), delta-9-tetrahydrocannabinol (THC), or a combination thereof.

### DETAILED DESCRIPTION

The present technology can be employed in any personal vaporizer that uses a liquid composition or base as a carrier so that oil-soluble compounds and/or resins can be loaded into the personal vaporizer and inhaled as a vapor.

When the vapor is inhaled by the person using the personal vaporizer, the vapor at least enters the person's mouth. Generally the vapor is drawn in as the person breathes, however the vapor may be carried by a propellant. In the present application, “inhale” means any breathe in, draw in, ingest, take into the mouth, trachea, and/or lungs.

Many over-the-counter and other specialty vaporizers use various forms of personal vaporizer liquid. The present liquid compositions could be used in any of those devices, and perhaps others that may be adapted for vaporizing non-liquid materials.

The present compositions comprise a liquid base that acts as a carrier for oil-soluble compounds and/or resins to be vaporized and inhaled. Liquid bases include propylene glycol, glycerol and PEG-400. Propylene glycol is presently preferred, as glycerol tends to be less effective in dissolving oils and less compatible with presently preferred emulsifiers, and PEG-400 has less evidence supporting its safety in personal vaporizers. The types and amounts of liquid base and lipid solubilizers should be selected so that a sufficiently stable emulsion is formed.

The present compositions comprise one or more “lipid solubilizers” which refers to compounds that solubilize or increase the solubility or dispersion of lipids or lipophilic material in hydrophilic compositions, including but not limited to aqueous compositions. In some embodiments, the present compositions may be non-aqueous compositions having a hydrophilic phase dispersed therein. Preferred lipid solubilizers are those which have been approved as food additives by one or more regulatory agencies such as the US Food and Drug Administration (“food-grade” solubilizers). Examples of preferred lipid solubilizers are mono- and diglycerides of fatty acids (E471 food additives), esters of mono- and diglycerides of fatty acids (E472a to E472f food additives), propane-1,2-diol esters of fatty acids (E477 food additives), and triethyl citrate (CAS#77-93-0; E1505 food additive). These lipid solubilizers improve the functional properties of a liquid composition for use in a personal vaporizer. Glycerol monolaurate (CAS#142-18-7) is a preferred E471 food additive. The lipid solubilizer can be a mixture of a monoglyceride or diglyceride of a fatty acid, such as a mixture containing two different monoglycerides (monoglycerides of different fatty acids), or a mixture of a monoglyceride of a fatty acid and a diglyceride of a fatty acid (where the fatty acids are the same or different), or two different diglyceride (with different fatty acids on one or both diglycerides). The lipid solubilizer can be a mixture of an ester of a monoglyceride or diglyceride of one or more fatty acids, such as a mixture containing two different esters of monoglycerides (esters of monoglycerides of different fatty acids), or a mixture of esters of a monoglyceride of a fatty acid and a diglyceride of a fatty acid (where the fatty acids are the same or different), or two different esters of diglyceride (with different fatty acids on one or both esters of diglycerides). The lipid solubilizer can be a mixture of a propane-1,2-diol ester of one or more fatty acids, such as a mixture of propane-1,2-diol esters containing different more fatty acids. The lipid solubilizer can be a mixture of a tri(C₁₋₈) alkyl ester of a C₂₋₈ organic acid or mixture thereof, such as a mixture of esters having different alkyl groups on the same or different compounds, and/or compounds made from the same or different organic acids.

The lipid solubilizer can be a monoglyceride of a fatty acid (including 1-monoacylglycerols and 2-monoacylglycerols), such as glycerol monolaurate (also referred to as monolaurin or 1-lauroylglycerol), 2-lauroylglycerol, 1-myristoylglycerol, 2-myristoylglycerol, 1-palmitoylglycerol, 2-palmitoylglycerol, 1-stearoylglycerol, 2-stearoylglycerol, 1-oleoylglycerol, 2-oleoylglycerol, 1-arachidoylglycerol, 2-arachidoylglycerol, 1-arachidonoylglycerol, 2-arachidonoylglycerol (which is also a ligand for cannabinoid receptors in the brain), 1-linoleoylglycerol, 2-linoleoylglycerol, 1-caproylglycerol, 2-caproylglycerol, 1-capriloylglycerol, and 2-capriloylglycerol. The lipid solubilizer can be a diglyceride of a fatty acid, such as 1,2-dilauroyl glycerol, 1,2-dipalmitoyl glycerol 1,2-dioleoyl glycerol, 1,2-distearoyl glycerol, 1-palmitoyl-2-oleoyl-glycerolor others. In some embodiments, each of the fatty acids is a C₆-C₂₂ fatty acid, alternatively a C₈-C₂₀ fatty acid, alternatively a C₁₀-C₁₈ fatty acid. The fatty acid moiety can be saturated or unsaturated. The lipid solubilizer can be a commercially available food additive, such as E471. E471 food additives are used as emulsifiers and comprise mono- and diglycerides of fatty acids.

The lipid solubilizer can be an ester of a monoglyceride or diglyceride of one or more fatty acids, such as caprylic and capric mono- and diglyceride esters, diacetyltartaric and other fatty acid esters of glycerol, or others. The lipid solubilizer can be a propane-1,2-diol ester of one or more fatty acids, such as propylene glycol heptanoate, propylene glycol monocaprylate, propylene glycol dilaurate, propylene glycol monocaprylate, propylene glycol monolaurate, or others.

The lipid solubilizer can be an alkyl or alkylenyl ester of a C₂₋₈ organic acid, including triethyl citrate, which is the commercially available food additive designated E1505. E1505 is used as a foam stabilizer. Other alkyl esters of fatty acids include trimethyl citrate, tripropyl citrate, tributyl citrate, or tri(C₁₋₈) citrate, where each of the three C₁₋₈ moieties can be the same or different. Other alkyl esters of fatty acids include tri(C₁₋₈) lactate, tri(C₁₋₈) acetate, tri(C₁₋₈) formate, and tri(C₁₋₈) oxalate.

The lipid solubilizer can be a C₁₋₅ alkyl or alkylenyl ester of glycerol, such as glyceryl triacetate (E1518 food additive) or a mixture thereof, such as a mixture of esters having different alkyl and/or alkylenyl groups on the same or different compounds.

The lipid solubilizer can be a mixture or combination of any of the foregoing components.

Suitable amounts of the lipid solubilizer (either individually or collectively where there is more than one) in a liquid composition include at least about 0.001% by weight, alternatively at least about 0.005% by weight, alternatively at least about 0.01% by weight, alternatively at least about 0.02% by weight, alternatively at least about 0.05% by weight, alternatively at least about 0.07% by weight, alternatively at least about 0.1% by weight, alternatively at least about 0.15% by weight, alternatively at least about 0.2% by weight, alternatively at least about 0.25% by weight, alternatively at least about 0.5% by weight, alternatively at least about 0.75% by weight, alternatively at least about 1% by weight, alternatively at least about 1.2% by weight, alternatively at least about 1.4% by weight, alternatively at least about 1.8% by weight, alternatively at least about 2% by weight, alternatively at least about 3% by weight, alternatively at least about 5% by weight. Suitable amounts of the lipid solubilizer (either individually or collectively where there is more than one) in a liquid composition include at most about 25% by weight, alternatively at most about 20% by weight, alternatively at most about 15% by weight, alternatively at most about 10% by weight, alternatively at most about 5% by weight, alternatively at most about 2.5% by weight, alternatively at most about 2% by weight, alternatively at most about 1.5% by weight, alternatively at most about 1% by weight, alternatively at most about 0.75% by weight, alternatively at most about 0.5% by weight, alternatively at most about 0.2% by weight. Any of the foregoing minimums can be combined with any of the foregoing maximums to provide a range, provided that the minimum is less than the maximum. For examples a suitable range is from about 0.01% by weight to about 1% by weight, alternatively from about 0.02% by weight to about 0.5% by weight, alternatively from about 0.05% by weight to about 0.2% by weight.

The foregoing liquid compositions can be used as a stock liquid composition into which a manufacturer or consumer places a desired amount of oil-soluble compounds and/or resins (such as medicinal agents, flavorants, or other components) to form a stable emulsion suitable for use in a personal vaporizer. The oil-soluble compounds and/or resins are added to a suitable amount of the stock liquid composition to form a liquid mixture. It may be desirable to heat and/or agitate the liquid mixture so that resin solids and oily liquids will disperse and form a stable emulsion appropriate for use in personal vaporizers. Temperatures for forming the stable emulsion from the liquid mixture are below the boiling point of the liquid base. When a consumer forms the stable emulsion, the temperatures will usually be substantially lower than the boiling point; for example, suitable temperatures can range from about 35° C. to about 90° C., alternatively from about 50° C. to about 70° C., alternatively about 60° C. It may also be desirable to stir, shake or otherwise agitate the liquid mixture in order to disperse the oil-soluble compounds and/or resins.

In some embodiments of the present invention, a stable emulsion containing oil-soluble compounds and/or resins in a liquid base is provided. The stable emulsion may be stored in a container suitable for delivery to a consumer. The stable emulsion may be provided in a vial, cartridge or other container. Preferably the container is air-tight. The stability of the emulsion may be measured or quantified by no visible separation after 30 days.

Among the oil-soluble compounds are cannabinoids and cannabis extracts (particularly cannabidiol (CBD), and/or delta-9-tetrahydrocannabinol (THC)), boswellic acid, shikimic acid, phellendrene, piperitone, citral, methyl cinnamate, geranyl acetate, linalool, linalyl acetate, eugenol, chavicol, safrole and estragole. Another example of an oil-soluble compound is oleamide. Oleamide is found in and can be extracted from Da Zao (chinese dates). Other herbal extracts can be mixed into the liquid compounds to allow vaporization of a wide range of medicinal compounds. Glycerin extracts from the manufacturer Gaia Herbs (Kava Kava, Ginseng, and Holy Basil) were easily mixed into the liquid compositions to form a stable emulsion containing the chosen herbal extract. Some glycerin separated during this preparation, as glycerin is somewhat incompatible in these formulas.

Cannabis extracts can be prepared by solvent extraction such as with ethanol, steam distillation, solvent-less extraction such as with propane or butane, or carbon dioxide extraction. Cannabis extracts can vary in color and viscosity. The extract can be diluted with edible oil for improved handling.

The present compositions can also include one or more resins. “Resins” refers to resins or resin extracts obtained from plants suitable for human inhalation. Resins includes extracts from plants such as cannabis or fruits such as Da Zao. Resins also includes boswellic acid, shikimic acid, frankincense, or myrrh.

Suitable amounts of the oil-soluble compound or resin (either individually or collectively where there is more than one) in a liquid composition include at least about 0.001% by weight, alternatively at least about 0.005% by weight, alternatively at least about 0.01% by weight, alternatively at least about 0.02% by weight, alternatively at least about 0.05% by weight, alternatively at least about 0.07% by weight, alternatively at least about 0.1% by weight, alternatively at least about 0.15% by weight, alternatively at least about 0.2% by weight, alternatively at least about 0.25% by weight, alternatively at least about 0.5% by weight, alternatively at least about 0.75% by weight, alternatively at least about 1% by weight, alternatively at least about 1.2% by weight, alternatively at least about 1.4% by weight, alternatively at least about 1.8% by weight, alternatively at least about 2% by weight, alternatively at least about 3% by weight, alternatively at least about 5% by weight, alternatively at least about 10% by weight, alternatively at least about 15% by weight, alternatively at least about 20% by weight. Suitable amounts of the oil-soluble compound or resin (either individually or collectively where there is more than one) in a liquid composition include at most about 90% by weight, alternatively at most about 85% by weight, alternatively at most about 80% by weight, alternatively at most about 75% by weight, alternatively at most about 72.5% by weight, alternatively at most about 70% by weight, alternatively at most about 67.5% by weight, alternatively at most about 65% by weight, alternatively at most about 60% by weight, alternatively at most about 50% by weight, alternatively at most about 40% by weight, alternatively at most about 33% by weight, alternatively at most about 30% by weight, alternatively at most about 25% by weight, alternatively at most about 20% by weight, alternatively at most about 15% by weight, alternatively at most about 10% by weight, alternatively at most about 5% by weight, alternatively at most about 2.5% by weight, alternatively at most about 2% by weight, alternatively at most about 1.5% by weight, alternatively at most about 1% by weight, alternatively at most about 0.75% by weight, alternatively at most about 0.5% by weight, alternatively at most about 0.2% by weight. Any of the foregoing minimums can be combined with any of the foregoing maximums to provide a range, provided that the minimum is less than the maximum. For examples a suitable range is from about 5% by weight to about 50% by weight, alternatively from about 5% by weight to about 33% by weight, alternatively from about 10% by weight to about 33% by weight, alternatively from about 20% by weight to about 33% by weight, alternatively from about 5% by weight to about 20% by weight, depending on the selection of liquid base, lipid, solubilizer(s) and cannabis extract.

In some currently preferred embodiments, the liquid composition further includes one or more oils or resins. The present compositions comprise one or more oil-soluble compounds in the liquid base (e.g. propylene glycol) containing one or more lipid solubilizers (e.g., monolaurin and triethyl citrate). The composition can be prepared with one or more of the lipid solubilizers. For example, in some embodiments, the liquid composition comprises propylene glycol and monolaurin; in other embodiments, the liquid composition comprises propylene glycol and triethyl citrate.

The liquid composition or stable emulsion can include additional components, such as flavorants, humectants, weighting agents, anti-oxidants and other components.

In some embodiments, the liquid composition includes one or more flavorants. The term “flavorant” as used herein refers to a compound that provides a desired taste and/or smell when vaporized. The flavorant can be a natural or artificial compound, and it can, but does not have to be, oil-soluble. Flavorants include isoamyl acetate (or other banana flavorant), benzaldehyde (or other almond flavorant), cinnamic aldehyde (or other cinnamon flavorant), citric acid or ethyl propionate (or other fruity flavorant), methyl anthranilate (or other grape flavorant), limonene (or other orange flavorant), ethyl decadienoate (or other pear flavorant), allyl hexanoate (or other pineapple flavorant), ethyl maltol (or other sugar or cotton candy flavorant), ethylvanillin (or other vanilla flavorant), methyl salicylate (or other wintergreen flavorant), glyceryl monoacetate (E1516 food additive), glyceryl diacetate (E1517 food additive), and combinations thereof.

Additional components that may be included in the liquid composition or stable emulsion are taurine, caffeine, glucuronolactone, tryptophan, gamma-aminobutyric acid, melatonin, epimedium, yohimbine, and others.

Suitable humectants include glyceryl triacetate (E1518 food additive).

Suitable weighting agents include glycerol ester of wood rosin, ester gum, dammar gum, and sucrose acetoisobutyrate (SAIB). Weighting agents are often used with low density materials in emulsions to adjust the density of the oil phase. For cannabis extracts that are very oily and have low density, it may be particularly desirable to add a weighting agent to a liquid composition intended for used in a personal vaporizer.

The liquid composition comprising the oil-soluble compounds or resin should have a density and viscosity suitable for use in a personal vaporizer. High viscosity significantly inhibits vaporizer performance, so lipid solubilizers with reduced thickening properties are essential. For example, a liquid composition having a density of about 100 cps, alternatively about 75 cps or less, alternatively about 50 cps or less, alternatively about 42 cps (the viscosity of propylene glycol) or more. The foregoing values can be combined to form a range. Viscosity values are determined at 25° C. using standard equipment for measuring viscosity of liquids.

The present liquid compositions provide stable emulsions for at least one month, often for least six months or nine months or twelve months. If the present compositions are to be stored for a significant amount of time, it is recommended that they be stored in sealed containers. If compositions of this type are exposed to air for a significant period, the emulsion becomes unstable. One reason for this is that propylene glycol is a humectant, a substance that pulls water vapor out of the air. As water is dissolved into the formula, it may displace emulsified waxes and essential oils, causing separation. When the present compositions are kept in closed containers, sealed tightly against exposure to air, they are stable as emulsions prevents this from happening, even after six, nine or twelve months of storage.

The present compositions and methods can be used for treating various diseases, disorders or conditions. For example, it is contemplated that the present compositions and methods, particularly when the composition includes one or more cannabinoids or cannabis extracts, can be used for treating pain such as cancer pain, bone pain, neuropathic pain and others; central nervous system disorders such as psychosis or schizophrenia; autoimmune diseases such as multiple sclerosis, inflammatory bowel disease; withdrawal or relapse from opioid dependence. The term “treating” as used herein can include any of alleviating, reducing, improving, mitigating, or eliminating a disease, disorder or condition.

An exemplary personal vaporizer will have a reservoir for holding the liquid composition to be vaporized, a heater, atomizer, nebulizer or other means for vaporizing in fluid connection with the reservoir, a power source such as a battery that provides power to the heater, atomizer, nebulizer or other means when activated, a vapor chamber where vapor is formed, a mouthpiece for a subject to inhale from the personal vaporizer, and a pathway from the vapor chamber to the mouthpiece. The vapor chamber and the reservoir may be separate spaces or may be the same space in the personal vaporizer. When the personal vaporizer is activated, the liquid composition in the reservoir is vaporized, and the user provides suction by inhalation to inhale the vaporized liquid composition.

The liquid composition can be loaded into the personal vaporizer in any suitable way. For some devices, the liquid composition will be poured into a reservoir of the personal vaporizer. For other devices, a cartridge that is pre-filled with the liquid composition may be attached to the personal vaporizer so as to provide the liquid composition to the reservoir or to the heater, atomizer or nebulizer. In some embodiments, the pre-filled cartridge may be the reservoir for the personal vaporizer.

### EXAMPLE 1

In this example, a liquid composition suitable for use as a stock emulsion was prepared. The composition was prepared by dissolving 0.1% by mass of an E471 food additive (monolaurin) and 0.1% by mass of E1505 (triethyl citrate) into 100 grams of propylene glycol. The mixture was stirred over low heat (about 60° C.). After 30 minutes, a stable emulsion was formed. The liquid was removed from heat and poured into storage containers.

The composition had the odor and appearance of typical personal vaporizer liquids (clear and odorless). No thermal decomposition products were detected when the emulsion was used in a personal vaporizer, in that no burning or smoking was observed, and the composition was suitable for forming a stable emulsion with the addition of oil-soluble compounds and/or resins, for use in a personal vaporizer. This procedure was repeated 5 times with consistent results.

### EXAMPLE 2

The liquid solution from Example 1 was combined with 1000 mg of cannabis extract for vaporization. The solution had the odor and appearance of typical personal vaporizer liquids (clear and odorless) and the solution formed a stable emulsion with the addition of oil-soluble compounds (in this case, cannabis extract). The solution was prepared by dissolving 1000 mg the desired amount of cannabis extract into 2.5 ml of the liquid solution. With mild heat and agitation, the cannabis extract was emulsified into an alcohol-free personal vaporizer liquid. This personal vapor liquid contained desired amounts of cannabis extracts in a stable emulsion for use in a personal vaporizer.

The liquid composition was an emulsion that remained stable for 30+days, as indicated by a visual separation test. No thermal decomposition products were detected when the emulsion was used in a personal vaporizer, in that no burning or smoking was observed, and the taste and potency of the vaporized material was not adversely affected by the food additives. This procedure was repeated 5 times, with consistent results.

### EXAMPLE 3

In this example, a series of liquid compositions were prepared and evaluated for their emulsion stability. Varying compositions were tested that utilized different lipid solubilizers (8 in total). The liquid base in these compositions was propylene glycol. Test samples (liquid compositions including cannabis extract) were subjected to a range of conditions: temperature, sunlight, extended storage, and varied ingredient concentration. Various types of cannabis extract were also tested. For each test sample, observations were noted about the color, viscosity, general visual appearance of each sample, and any changes seen over time.

Materials and Methods: Testing was performed in 20 ml glass scintillation vials. Mixing of the liquid base and lipid solubilizer was done entirely in borosilicate glassware, and ingredients were measured by mass using an AND EJ-610 balance. Samples were heated using a Scilogex MS7-H55O digitally controlled hot plate. Cannabis extract was produced using an Apeks closed-system solvent extractor running instrument grade propane. Extracts were also purchased from Organic Alternatives, a medical marijuana dispensary in Fort Collins Colo. Lipid solubilizers were purchased from Vigon International, Abitech Corporporation, and Lauricidin. From Abitech's Campul product line: Propylene Glycol Monolaurate, CAS#27194-74-7 (PG-12); Propylene Glycol Caprylate, CAS#31565-12-5 and 7384-98-7 (PG-8); Propylene Glycol Dilaurate, CAS#22788-19-8 (PG-2L); Glyceryl Monooleate, CAS#25496-72-4 (GMO-50); Glyceryl Caprylate, CAS#02640226-6 (MCM C8). From Vigon International: Triethyl Citrate, CAS#: 77-93-0 (TEC); Triacetin, CAS#: 102-76-1 (TA). From Lauricidin: Glyceryl Monolaurate (monolaurin), CAS#: 142-18-7 (GML). Five of the tested emulsifiers are chemicals in the category E471 (glycerol fatty acid esters) and E477 (propylene glycol fatty acid esters). These five, along with three 3 other additives (glyceryl monolaurate, triethyl citrate, and triacetin), were tested with various types of cannabis extracts at various concentrations and in various environmental conditions.

Formula preparation: Formulas using lipid solubilizers were prepared in three concentrations: 0.1%, 0.5% and 1.0% by mass, in a solvent base of propylene glycol. Formulas were prepared by mixing one of the emulsifying additives into propylene glycol on a hot plate at 65° C. until all solids had dissolved. Stock solutions were made of each of the eight lipid solubilizers at each of the three concentrations, producing a total of 24 different formulas at a quantity of approximately 500 ml each. By way of example, 500 ml of 1.0% solution was prepared by adding 5.00 grams of lipid solubilizer to 495.00 grams of propylene glycol in a glass beaker. The mixture was then heated and stirred until all solids were dissolved and the solution was completely clear. After cooling the formula was ready for use. Table 1 summarizes the formulas:

TABLE 1

| | Lipid | |
|---|---|---|
| Formula | Solubilizer | Conc. |
| A | PG-12 | 0.1% |
| B | PG-8 | 0.1% |
| C | PG-2L | 0.1% |
| D | GMO-50 | 0.1% |
| E | MCM C8 | 0.1% |
| F | GML | 0.1% |
| G | TEC | 0.1% |
| H | TA | 0.1% |
| I | PG-12 | 0.5% |
| J | PG-8 | 0.5% |
| K | PG-2L | 0.5% |
| L | GMO-50 | 0.5% |
| M | MCM C8 | 0.5% |
| N | GML | 0.5% |
| O | TEC | 0.5% |
| P | TA | 0.5% |
| Q | PG-12 | 1.0% |
| R | PG-8 | 1.0% |
| S | PG-2L | 1.0% |
| T | GMO-50 | 1.0% |
| U | MCM C8 | 1.0% |
| V | GML | 1.0% |
| W | TEC | 1.0% |
| X | TA | 1.0% |

Extract types: To prepare liquid compositions from the formulas above, two cannabis extracts were used. One was a lighter waxy (firm) extract made from cannabis material from the strain “Bubba Kush.” (Extract A). The other was a darker oilier (soft) extract made from material of lower quality and of unknown strain obtained from Colorado caregiver material (Extract B). Throughout the testing period various small tests were performed on extract material of a wide range in quality, but our two main types of extract mentioned above were used for all test conditions.

Test Sample preparation: For each sample, the appropriate quantity of cannabis extract and liquid formula were added to a vial. The vial was placed on a hot plate at 85° C. until the extract melted. The vial was gently shaken to disperse the extract. Each of the formula recipes were tested with five concentrations of extract: 5%, 10%, 20%, 33%, and 50%, by mass. For example: to produce a 10% extract sample we placed 9.00 grams of one of the stock oil emulsifier formulas, and 1.00 grams of one of cannabis extracts in a 20 ml scintillation vial and applied heat. The components remained separate until heat and agitation are applied.

Wax layer separation: When the extract was heated in the formula, some components of the extract were not stable in the emulsion and rose to the surface forming a thin waxy layer on top. These compounds are undesirable, waxy compounds that can be easily removed once cooled. When the formula and extract are heated, the extract disperses with agitation. If allowed to sit on a heat source for a few extra minutes after dispersal, the components of the extract that are not stable in the emulsion will rise to the surface. This is typically referred to as “creaming” in the food and beverage industry. This wax layer on top hardens once cooled and can be easily removed or separated from the liquid emulsion. If this layer is not allowed to separate and harden, small bits of this waxy layer can precipitate out of the emulsion later on, interfering with vaporizer performance. The waxy layer can be removed by vacuum filtration. In testing of the liquid and wax portions, it was determined that there were about 6 to about 8% cannabinoids, whereas the liquid had about 18% cannabinoids.

Formula Concentration: All of the liquid compositions formed stable emulsions. A concentration of 0.1% of the liquid solubilizer appeared to be adequate for a stable emulsion as there was no apparent difference between the three different liquid solubilizer concentrations in initial stability tests; after several days, all the compositions remained stable. Accordingly, a lipid solubilizer concentration of 0.1% was selected for subsequent testing, since a lower concentration of lipid solubilizer is beneficial in terms of lower usage of material and less impact on flavor. Liquid compositions containing 0.1% of liquid solubilizer were sample #1-80 (described in Table 3). Additional formulas were also developed containing 0.1% GML along with one or both of triethyl citrate and triacetin, as set forth in Table 2 (the balance of each formula was proplyele glycol):

TABLE 2

| | Conc. of Lipid | Conc. of | Conc. of |
|---|---|---|---|
| Formula | Solubilizer | TEC | TA |
| AA | 0.1% GML | 0.02% | 0.02% |
| BB | 0.1% GML | — | 0.02% |
| CC | 0.1% GML | 0.02% | — |
| DD | 0.1% MCM C8 | 0.02% | 0.02% |

The viscosity of each sample is rated, in which “1” indicates lower viscosity, “2” indicates moderate viscosity, and “3” indicates higher viscosity.

TABLE 3

| Sample | | | | Viscosity |
|---|---|---|---|---|
| ID | Extract Type | Extract Conc. | Formula | Rating |
| 1 | Bubba Kush | 5% | A | 3 |
| 2 | Bubba Kush | 5% | B | 2 |
| 3 | Bubba Kush | 5% | C | 2 |
| 4 | Bubba Kush | 5% | D | 2 |
| 5 | Bubba Kush | 5% | E | 1 |
| 6 | Bubba Kush | 5% | F | 1 |
| 7 | Bubba Kush | 5% | G | 2 |
| 8 | Bubba Kush | 5% | H | 2 |
| 9 | Bubba Kush | 10% | A | 3 |
| 10 | Bubba Kush | 10% | B | 2 |
| 11 | Bubba Kush | 10% | C | 2 |
| 12 | Bubba Kush | 10% | D | 2 |
| 13 | Bubba Kush | 10% | E | 1 |
| 14 | Bubba Kush | 10% | F | 1 |
| 15 | Bubba Kush | 10% | G | 2 |
| 16 | Bubba Kush | 10% | H | 2 |
| 17 | Bubba Kush | 20% | A | 3 |
| 18 | Bubba Kush | 20% | B | 2 |
| 19 | Bubba Kush | 20% | C | 2 |
| 20 | Bubba Kush | 20% | D | 2 |
| 21 | Bubba Kush | 20% | E | 1 |
| 22 | Bubba Kush | 20% | F | 1 |
| 23 | Bubba Kush | 20% | G | 2 |
| 24 | Bubba Kush | 20% | H | 2 |
| 25 | Bubba Kush | 33% | A | 3 |
| 26 | Bubba Kush | 33% | B | 3 |
| 27 | Bubba Kush | 33% | C | 2 |
| 28 | Bubba Kush | 33% | D | 2 |
| 29 | Bubba Kush | 33% | E | 1 |
| 30 | Bubba Kush | 33% | F | 1 |
| 31 | Bubba Kush | 33% | G | 2 |
| 32 | Bubba Kush | 33% | H | 2 |
| 33 | Bubba Kush | 50% | A | 3 |
| 34 | Bubba Kush | 50% | B | 3 |
| 35 | Bubba Kush | 50% | C | 3 |
| 36 | Bubba Kush | 50% | D | 2 |
| 37 | Bubba Kush | 50% | E | 2 |
| 38 | Bubba Kush | 50% | F | 2 |
| 39 | Bubba Kush | 50% | G | 2 |
| 40 | Bubba Kush | 50% | H | 2 |
| 41 | Dark Oil | 5% | A | 3 |
| 42 | Dark Oil | 5% | B | 2 |
| 43 | Dark Oil | 5% | C | 2 |
| 44 | Dark Oil | 5% | D | 2 |
| 45 | Dark Oil | 5% | E | 1 |
| 46 | Dark Oil | 5% | F | 1 |
| 47 | Dark Oil | 5% | G | 2 |
| 48 | Dark Oil | 5% | H | 2 |
| 49 | Dark Oil | 10% | A | 3 |
| 50 | Dark Oil | 10% | B | 2 |
| 51 | Dark Oil | 10% | C | 2 |
| 52 | Dark Oil | 10% | D | 2 |
| 53 | Dark Oil | 10% | E | 1 |
| 54 | Dark Oil | 10% | F | 1 |
| 55 | Dark Oil | 10% | G | 2 |
| 56 | Dark Oil | 10% | H | 2 |
| 57 | Dark Oil | 20% | A | 3 |
| 58 | Dark Oil | 20% | B | 2 |
| 59 | Dark Oil | 20% | C | 2 |
| 60 | Dark Oil | 20% | D | 2 |
| 61 | Dark Oil | 20% | E | 1 |
| 62 | Dark Oil | 20% | F | 1 |
| 63 | Dark Oil | 20% | G | 2 |
| 64 | Dark Oil | 20% | H | 2 |
| 65 | Dark Oil | 33% | A | 3 |
| 66 | Dark Oil | 33% | B | 3 |
| 67 | Dark Oil | 33% | C | 2 |
| 68 | Dark Oil | 33% | D | 2 |
| 69 | Dark Oil | 33% | E | 1 |
| 70 | Dark Oil | 33% | F | 1 |
| 71 | Dark Oil | 33% | G | 2 |
| 72 | Dark Oil | 33% | H | 2 |
| 73 | Dark Oil | 50% | A | 3 |
| 74 | Dark Oil | 50% | B | 3 |
| 75 | Dark Oil | 50% | C | 3 |
| 76 | Dark Oil | 50% | D | 2 |
| 77 | Dark Oil | 50% | E | 2 |
| 78 | Dark Oil | 50% | F | 2 |
| 79 | Dark Oil | 50% | G | 2 |
| 80 | Dark Oil | 50% | H | 2 |
| 81 | Bubba Kush | 20% | AA | 1 |
| 82 | Bubba Kush | 20% | BB | 1 |
| 83 | Bubba Kush | 20% | CC | 1 |
| 84 | Bubba Kush | 20% | DD | 1 |
| 85 | Dark Oil | 20% | AA | 1 |
| 86 | Dark Oil | 20% | BB | 1 |
| 87 | Dark Oil | 20% | CC | 1 |
| 88 | Dark Oil | 20% | DD | 1 |

All the liquid compositions of samples 1 through 88 remained stable emulsions for at least a month, without visible separation of components. The liquid compositions were in sealed vials, protected from exposure to air. The flavor of the samples was not tested and may not be preserved, but it is a substantial advance to be able to provide emulsions of oil-soluble compounds such as cannabis extracts having this degree of stability.

Extract types: The lighter extract (Bubba Kush) often produced a clean looking, more transparent emulsion, while the darker less viscous extract seemed to produce a more opaque emulsion and produced significantly more waxy precipitate.

Extract Concentration: There appeared to be no difference in emulsion stability between extract concentrations, they all were equally stable, but viscosity was clearly affected. Higher concentrations of extract produced higher viscosities. A single sample of 75% extract was tested and the emulsion was stable, but the solution was very viscous.

Emulsifying additives: The emulsifiers each performed similarly in forming stable emulsions of oil-soluble compounds in propylene glycol, with the most noticeable differences being variation in viscosity. PG-12 produced the most viscous solution, while the MCM C8 produced the least viscosity. This observed variation in viscosity indicates that glycerol fatty-acid esters are superior at producing low-viscosity cannabis emulsions in propylene glycol when compared with propylene glycol fatty-acid esters.

Solubilizers: Two other ingredients of a different category, triethyl citrate and triacetin, were also tested. These ingredients appeared to perform adequately for the purpose of extract emulsification. These two ingredients could be useful as viscosity reducers in a formula, acting as secondary emulsifiers or co-solvents, and influencing the properties of the final product. However, they each impart a distinct and somewhat unpalatable sensation and flavor to the final product, so their concentrations must be limited to prevent them from interfering with the final product experience. It is contemplated that the present liquid compositions preferably have triethyl citrate and triacetin in concentrations of about 0.05% or less, alternatively about 0.02% or less.

Monolaurin with Triethyl Citrate and Triacetin: Monolaurin (glyceryl monolaurate) is an effective and flavorless emulsifier for the purpose of this formula. Triethyl citrate and triacetin can be used in addition, to decrease final product viscosity and allow for better vaporizer performance. With the addition of 0.02% of both triethyl citrate and triacetin, the solution was slightly less viscous yet the concentration of these two additives was low enough that no changes to flavor or aroma were observed.

Sun exposure: Exposure to sunlight caused rapid degradation of the emulsified extract. After a few weeks of all-day direct sun the sample became much darker than its original color and signs of separation had begun, presumably caused by decomposition of the extract components, not degradation of the emulsifier ingredients.

Temperature: Freezing temperatures did not disrupt the emulsion, nor did higher temperatures up to 100° C. Some waxy components of the extract can separate at higher temperatures; it is important to remove these components during the initial emulsification process so they do not precipitate later on during periods of increased heat. At extremely high temperatures (300° C. and above) the extract begins to degrade rapidly and additional separation can occur.

Shelf Life: The test samples (all eight emulsifier formulas with all five extract concentrations of both types of extract, plus some variations, for a total a total of 88 samples) were prepared and evaluated for shelf life. Since their preparation, each vial was stored in a room away from direct sunlight and at approximately 21° C. After six months of storage, all of the test vials still showed emulsion stability. No separation or significant changes to the appearance was observed, including the solvent-less oil samples and the 75% extract sample.

### EXAMPLE 4a

Two types of solvent-less oil cannabis extracts were tested, and differences between propane extracts and butane extracts were examined. Two types of solvent-less oil (propane and butane) were tested in liquid compositions containing propylene glycol and 0.1% by weight monolaurin. It was observed that propane extract of cannabis tends to produce significantly more of the waxy precipitate than an equivalent quantity of butane extract. This could be due to a difference in propane's solvating capacity of waxier substances compared with butane. However, propane extracts may be preferred because they have a higher yield, better flavor, lack of toxicity, and are easier to remove from concentrate. Those emulsions were stable, though they exhibited a significant amount of sediment on the bottom of the vial. This is to be expected with solvent-less oil as it contains significant quantities of insoluble plant matter (e.g. cellulose). superfreeze in dry ice, then shake over filter, obtain oil dust. Purchased

### EXAMPLE 4b

A variety of cannabis extracts were also tested in small quantities in a liquid composition containing monolaurin at 0.1% in propylene glycol; “shatter” type extracts, and highly priced Cannabis Cup award winning extracts from the “Skywalker” and “Bruce Banner” cannabis strains, more specifically a butane hash oil of the Skywalker and Bruce Banner strains. “Shatter” is one type of extract that is provided as a glassy solid which can be shattered. Another type of extract is referred to as “budder”. Either type can be put into the present liquid compositions. All of these extracts showed good emulsion stability over a minimum of 4 weeks in a liquid composition comprising 0.1% by weight monolaurin and proplyene glycol as the balance of the liquid composition.

### EXAMPLE 4c

Some obscure extracts obtained from Colorado caregivers of very low quality had a much larger amount of waxy precipitate that was more difficult to remove. In cases where the extract was exceptionally dark and less viscous, the final product showed partial separation, a cloudy waxy layer on top that would not harden once cooled. Beneath the cloudy waxy layer was often a clearer emulsion with good vaporizer performance despite any bad taste of the extract.

### EXAMPLE 5

A liquid composition was prepared comprising a compound called oleamide, which is an amide of the fatty acid oleic acid. It is found in the brains of mammals and in various plants, and it is considered an endogenous cannabinoid. It is being studied as a potential medical treatment for mood and sleep disorders, and cannabinoid-regulated depression. Oleamide is very waxy and much more difficult of a substance to emulsify in propylene glycol than cannabis extract. However, acceptable emulsion stability can be achieved using the lipid solubilizers disclosed herein and the addition of one new one. A stable mixture (a liquid composition comprising oleamide) comprised 87.85% propylene glycol; 2.0% monolaurin; 10.0% triethyl citrate; 0.10% glycerol ester of wood rosin; and 0.5% oleamide. Glycerol ester of wood rosin is used in Gatorade® to stabilize citrus flavor oils. This liquid composition was suitable for use in a personal vaporizer.

All of the references cited herein, including patents, patent applications, and publications, are hereby incorporated in their entireties by reference.

In the present disclosure, wherever the word “comprising” is found, it is contemplated that the words “consisting essentially of” or “consisting of” may be used in its place. All percentages are weight percentages based on the total weight of the composition, unless otherwise indicated.

While particular elements, embodiments and applications of the present invention have been shown and described, it will be understood, of course, that the invention is not limited thereto since modifications can be made by those skilled in the art without departing from the scope of the present disclosure, particularly in light of the foregoing teachings.

What we claim is:

1. A liquid composition for a personal vaporizer comprising:
a liquid base suitable for vaporization and inhalation;
a lipid solubilizer selected from the group consisting of a monoglyceride or diglyceride of one or more fatty acids or mixture thereof an ester of a monoglyceride or diglyceride of one or more fatty acids or mixture thereof; a propane-1,2-diol ester of one or more fatty acids or mixture thereof; a tri(C₁₋₈) alkyl ester of a C₂₋₈ organic acid or mixture thereof; a C₁₋₅ alkyl or alkylenyl ester of glycerol or mixture thereof; and combinations thereof

2. The liquid composition of claim 1, wherein the liquid composition comprises one or more of said monoglyceride of one or more fatty acids or mixture thereof

3. The liquid composition of claim 2, wherein the liquid composition comprises from about 0.01% by weight to about 1% by weight of one or more of said monoglyceride of one or more fatty acids or mixture thereof

4. The liquid composition of claim 2, wherein the liquid composition comprises 1-lauroylglycerol.

5. The liquid composition of claim 1, wherein the liquid composition comprises a tri(C₁₋₈) alkyl ester of a C₂₋₈ organic acid.

6. The liquid composition of claim 5, wherein the liquid composition comprises triethyl citrate.

7. The liquid composition of claim 1, wherein the liquid base is propylene glycol, the lipid solubilizer comprises 1-lauroylglycerol, and the liquid composition further comprises triethyl citrate, triacetin or a mixture thereof.

8. The liquid composition of claim 7, wherein the liquid composition comprises no more than about 0.05% by weight of triethyl citrate, triacetin or a mixture thereof.

9. The liquid composition of claim 1, wherein the liquid composition is essentially free of ethanol.

10. A liquid composition comprising:
a liquid base suitable for vaporization and inhalation;
a lipid solubilizer selected from the group consisting of a monoglyceride or diglyceride of a fatty acid or mixture thereof; an ester of a monoglyceride or diglyceride of one or more fatty acids or mixture thereof; a propane-1,2-diol ester of one or more fatty acids or mixture thereof; a tri(C₁₋₈) alkyl ester of a C₂₋₈ organic acid or mixture thereof; a C₁₋₅ alkyl or alkylenyl ester of glycerol or mixture thereof; and combinations thereof; and
one or more oil-soluble compounds or resins or combinations thereof;
wherein the liquid composition is a stable emulsion.

11. The liquid composition of claim 10, wherein said one or more oil-soluble compounds comprises a flavorant or a medicinal agent.

12. The liquid composition of claim 11, wherein the medicinal agent comprises a cannabinoid, a cannabis extract or a combination thereof.

13. The liquid composition of claim 11, wherein the medicinal agent comprises cannabidiol (CBD), delta-9-tetrahydrocannabinol (THC), or a combination thereof.

14. A method of administering vaporized oil-soluble compounds and resins to a subject by inhalation, the method comprising:
vaporizing the liquid composition of claim 10 in a personal vaporizer.

15. The method of claim 14, further comprising dispensing the vaporized oil-soluble compounds or resins through a mouthpiece of the personal vaporizer.

16. The method of claim 14, further comprising inhalation of the vaporized oil-soluble compounds or resins.

17. The method of claim 14, further comprising treating a disease, disorder or condition with the vaporized oil-soluble compounds or resins.

18. A container holding a liquid composition comprising a liquid base suitable for vaporization and inhalation; a lipid solubilizer selected from the group consisting of a monoglyceride or diglyceride of one or more fatty acids or mixture thereof; an ester of a monoglyceride, or diglyceride of one or more fatty acids or mixture thereof; a propane-1,2-diol ester of one or more fatty acids or mixture thereof; a tri(C₁₋₈) alkyl ester of a C₂₋₈ organic acid or mixture thereof; a tri(C₁₋₈) alkyl ester of a C₂₋₈ organic acid or mixture thereof; a C₁₋₅ alkyl or alkylenyl ester of glycerol or mixture thereof; and combinations thereof; and one or more oil-soluble compounds or resins; wherein the liquid composition is a stable emulsion, wherein the container is adapted for attachment to a personal vaporizer.

19. A kit comprising a personal vaporizer, dispensary vessels, a battery charger, and the container of claim 18.

20. A kit comprising a personal vaporizer, dispensary vessels, and the container of claim 18.
